(19) United States
(12) Patent Application Publication
(10) Pub. No.: US 2020/0038220 A1
(43) Pub. Date: Feb. 6, 2020

(54) MODULAR ANKLE BRACE SYSTEM

(71) Applicant: Pro-Tech Brace & Equipment, LLC, Tampa, FL (US)

(72) Inventor: Ramoned Tereze Morrow, Tampa, FL (US)

(21) Appl. No.: 16/526,579

(22) Filed: Jul. 30, 2019

Related U.S. Application Data
(60) Provisional application No. 62/713,870, filed on Aug. 2, 2018.
(60) Provisional application No. 62/832,392, filed on Apr. 11, 2019.

(51) Int. Cl. A61F 5/01

(52) U.S. Cl. CPC A61F 5/0127; A61F 2005/0132

(57) ABSTRACT

The Modular Ankle Brace System discloses ankle braces that utilize modular construction and dual-hinge connections to provide support for the ankle while allowing for regular dorsiflexion and plantarflexion movements. The modular construction of the braces allow the braces to be used at various stages of recovery from an ankle injury or as a preventive brace to protect the ankle from rotation and twisting during physical activity. The components of the braces are connected with hinges that allow for support of the ankle without bulky wraps or braces over the ankle and underneath the heel. Thus, providing a more comfortable and functional ankle brace.

### CROSS-REFERENCE TO RELATED APPLICATION

This application claims the benefit of U.S. Provisional Patent App. No. 62/713,870 filed on Aug. 2, 2018, titled Ankle and Subtalar Joint Brace, and U.S. Provisional Patent App. No. 62/832,392 filed on Apr. 11, 2019, titled Dorsum Ankle and Subtalar Joint Brace, the disclosures of which are hereby incorporated by reference.

### FIELD

This application relates to the field of ankle and foot orthotics and, more particularly, to new designs for ankle braces that can be used to prevent and reduce the incidence of ankle sprains in sports as well as provide a modular orthotic for use in multiple stages of recovery for injured or surgically repaired ankles.

### BACKGROUND

Most ankle braces on the market focus on the ankle joint (between the tibia and talus). However, the majority of ankle sprains occur at the subtalar joint, (between the talus and calcaneus) straining the anterior talofibular ligament. The anterior talofibular ligament is one of the most frequently injured ligaments of the ankle and is the most frequently observed injury in the emergency room. Because of its lateral position in the ankle, the anterior talofibular ligament absorbs most of the negative impact when the foot is planted unnaturally or when the ankle twists and inverts in an awkward way.

What is needed is an ankle brace that focuses on bracing the most common area where ankle sprains occur, which is at the subtalar joint, and prevents sprains during common movements that result in sprains, such as plantarflexion and dorsiflexion.

### SUMMARY

The Modular Ankle Brace System may be used as an immobilizer at the onset of a serious ankle injury, then to be used during the recovery or rehabilitation stage and after recovery it may be configured to be used as a preventive measure from future sprains during active events. Ankle braces according to the Modular Ankle Brace System of the present disclosure allow the ankle to move with the without restricting plantarflexion and dorsiflexion but restricts inversion and eversion to protect the anterior talofibular ligament.

The rigid pieces or shells of the Modular Ankle Brace System may have a slim-line contoured shape to reduce space between the brace and the body for better support and better fit on the body and in a shoe. The braces may also include slip-resistant liners to secure the braces to the ankle and foot.

In one aspect of the Modular Ankle Brace System, a connector rigidly secures one rigid pieces or shell over the ankle and another rigid shell over the anterior lateral aspect of the foot. In some embodiments, the connector is a bracket. In some embodiments, the bracket may be hingedly connected to both rigid shells.

In another embodiment of the Modular Ankle Brace System, a brace comprises a two-piece rigid shell that is secured to the lateral area of the ankle and foot by a removable lining.

In another embodiment of the Modular Ankle Brace System, brackets are located on both sides of the ankle which allows for plantarflexion, dorsiflexion, and movement with respect to the changing transverse axis of the ankle to constantly protect and resist awkward twisting of the foot.

In another embodiment of the Modular Ankle Brace System, a rigid piece, or shell or cuff, is located on the anterior area of the leg and another rigid piece is located on dorsum of the foot to brace both sides of the ligamentous structure of the ankle.

In another embodiment, the Modular Ankle Brace System comprises a lateral piece positioned over a lateral aspect of an ankle and secured to the ankle by a strap; a support positioned over an anterior lateral aspect of a foot and secured to the foot and around the bottom of the mid-foot; and a connector having a first end and a second end, the first end of the bracket attached to the lateral piece and the second end attached to the support.

In another embodiment, the connector is a rigid bracket and a first end of the bracket is hingedly attached to the lateral piece and a second end of the bracket is hingedly attached to the support thereby creating a dual hinge relationship between the lateral piece and the support.

In another embodiment of the Module Ankle Brace System, a subtalar ankle brace is attached to the outside of a lower leg and foot to prevent rotation of an subtalar region of a foot while allowing regular flexion of the ankle. In an exemplary embodiment, the subtalar brace comprises a lateral cuff secured to the outside of the ankle; a support cuff secured to the anterior lateral aspect of the foot; and connector hingedly attached to the lateral cuff and the support cuff. In some embodiments, the connector may be a bracket, one end of the bracket is hingedly connected to the lateral cuff in a lower forward area and a second end of the bracket is hingedly connected to a rear and upper area of the support cuff.

In another embodiment of the Modular Ankle Brace System, a first support cuff is positioned over the dorsum aspect of the foot and is secured to a second support positioned on the anterior aspect of the leg by two dual hinges. One end of each hinge attaches on the lower lateral and medial sides of the second support cuff and the other end of the hinges attaches on the upper lateral and medial sides of the first support cuff.

In another embodiment of the Modular Ankle Brace System, a brace for supporting the subtalar ligaments comprises an ankle support and a foot support positioned over the lower third of the anterior portion of the leg.

In another embodiment of the Modular Ankle Brace System, an ankle and foot support with one front positioned stirrup used to brace both sides of the ankle with independent lateral and medial dual hinges.

In one aspect of the Modular Ankle Brace System, rigid pieces or shells of the supports or cuffs may be used with interchangeable liners, which allows a user to vary different liners with different cuffs, or supports, to obtain variable support or fit of an ankle brace according to the system.

In another aspect of the Modular Ankle Brace System, an ankle and foot support brace the interchangeable lining is made of a washable fabric.

In another aspect of the Modular Ankle Brace System, the supports, or cuffs or shells, may be provided as plastic with low melting temperature for easy custom shaping with fittings and attachments by the end user.

### BRIEF DESCRIPTION OF THE DRAWINGS

FIG. 1 is a right-side view of a right foot wearing a subtalar brace according to the Modular Ankle Brace System with liner straps open;

FIG. 2 is a right-side view of a right foot wearing a subtalar brace according to the Modular Ankle Brace System with liner straps closed;

FIG. 3 is an interior perspective view of a right foot wearing a subtalar brace according to the Modular Ankle Brace System;

FIG. 4 is a rear perspective view of a right foot being flexed in the plantar direction wearing a subtalar brace according to the Modular Ankle Brace System;

FIG. 5 illustrates the parts of a subtalar brace according to the Modular Ankle Brace System;

FIG. 6 illustrates a subtalar brace and a lateral extension according to the Modular Ankle Brace System;

FIG. 7 is a right-side view of a right foot wearing a subtalar brace with a lateral extension according to the Modular Ankle Brace System;

FIG. 8 illustrates the parts of a dorsum brace according to the Modular Ankle Brace System;

FIG. 9 illustrates an assembled dorsum brace according to the Modular Ankle Brace System;

FIG. 10 is a perspective view of both feet of a person wearing dorsum braces according to the Modular Ankle Brace System;

FIG. 11 is a rear view of a right foot wearing a brace according to the Modular Ankle Brace System;

FIG. 12 is a view of the bottom of a foot wearing a brace according to the Modular Ankle Brace System;

FIG. 13 illustrates the parts of an upper tibial cuff for use with a dorsum brace of the Modular Ankle Brace System;

FIG. 14 illustrates a dorsum brace and an upper tibial cuff being applied to a left foot and leg of a person according to the Modular Ankle Brace System;

FIG. 15 illustrates a left foot wearing a dorsum brace and an upper tibial cuff according to the Modular Ankle Brace System;

FIG. 16 illustrates an immobilizer for use with a brace of the Modular Ankle Brace System;

FIG. 17 is a right side view of a right foot with an immobilizer being applied to a dorsum brace according to the Modular Ankle Brace System;

FIG. 18 is a front perspective view thereof with straps open;

FIG. 19 is a front perspective view thereof with straps closed;

FIG. 20 illustrates a tension strap for use with the Modular Ankle Brace System;

FIG. 21 illustrates a tension strap being fastened to the bottom of a foot wearing a brace according to the Modular Ankle Brace System;

FIG. 22 illustrates a first method for applying a tension strap according to the Modular Ankle Brace System;

FIG. 23 illustrates a second method for applying a tension strap according to the Modular Ankle Brace System;

FIG. 24 illustrates a third method for applying a tension strap according to the Modular Ankle Brace System; and

FIG. 25 illustrates a fourth method for applying a tension strap according to the Modular Ankle Brace System.

### DETAILED DESCRIPTION

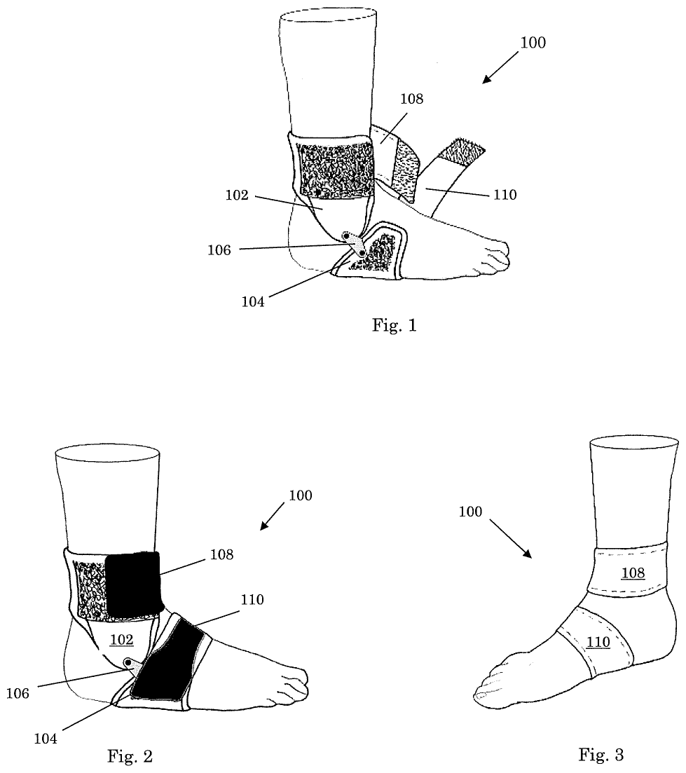

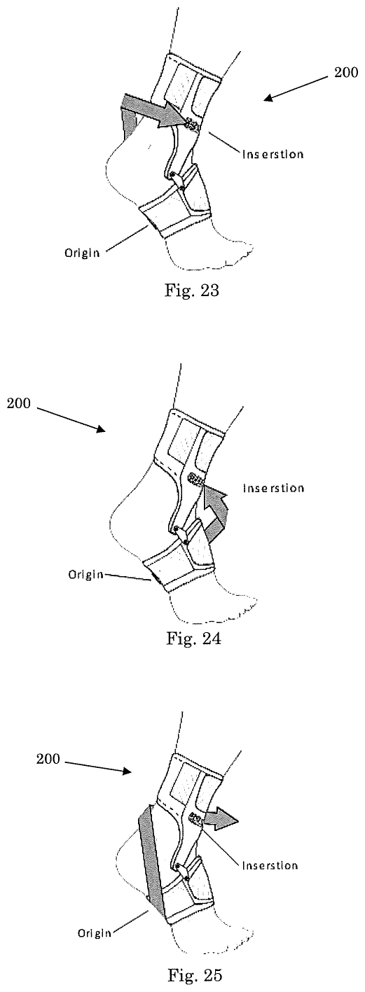

Referring to FIGS. 1-25, the Modular Ankle Brace System includes various embodiments that may be used with various attachments so that a single ankle brace may be used for stabilizing a user's ankle at various stages of recovery from an ankle injury. Alternatively, the same brace may be used without any of the attachments as a low-profile ankle support to protect against injury during physical activity. One embodiment of the Modular Ankle Brace System may include a lateral piece hingedly connected to an anterior-lateral foot support by a rigid bracket. Another embodiment of the Modular Ankle Brace System may include a tibial cuff hingedly connected to a dorsum cuff by two brackets.

Referring now to FIG. 1, a view of the lateral side of a right foot with a subtalar ankle brace 100 according to the Modular Ankle Brace System is shown. The subtalar ankle brace 100 is placed in position with the lateral piece 102 on the lateral aspect of the lower leg and the anterior lateral foot support 104 over the subtalar region of the foot. A connector hingedly joins to both the lateral piece 102 and the support 104, thereby creating a dual hinge connection.

In one exemplary embodiment, the connector may be a bracket 106. One end of the bracket 106 is attached to the lower forward part of the lateral piece 102 and the other end of the bracket 106 is attached to the support 104. The dual-hinged connection between the lateral piece 102 and the support 104 allows for full plantarflexion and dorsiflexion of the changing transverse axis of the ankle when walking or running. However, the subtalar ankle brace 100 restricts twisting and awkward rotation of the lateral piece 102 with respect to the support 104 during ankle inversion or eversion, thereby restricting twisting or rotation of the ankle.

In one embodiment, the subtalar brace 100 also includes a lateral liner 108 and a support liner 110. The liners 108, 110 may be made of flexible material to secure the subtalar brace 100 to a person. The lateral liner 108 attaches to the lateral piece 102 and the strap of the lateral liner 108 wraps around the lower leg to fasten the lateral piece 102. The support liner 110 attaches to the support 104 and the strap of the support liner 110 wraps around the mid-foot to fasten the support 104 against the anterior lateral aspect of the foot, as shown in FIG. 2.

In some embodiments of the Modular Ankle Brace System, the liners 108, 110 may be used with different braces.

FIG. 3 is an interior perspective view of the same right foot but showing the lateral liner 108 and the support liner 110 wrapped around the lower leg and the foot, respectively.

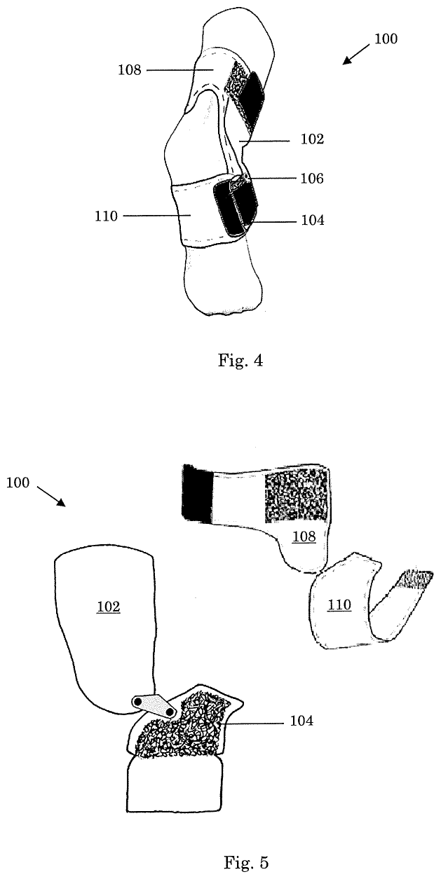

FIG. 4 illustrates a rear perspective view of the subtalar brace 100 showing the exposed heel and illustrating how the placement of the support 104 on the anterior lateral aspect of the foot allows for flexion of the ankle.

Referring now to FIG. 5, the parts of the subtalar brace 100 are shown. In one embodiment, lateral piece 102 and support 104 may be made of substantially rigid material, such as plastic or metal. In another embodiment, lateral piece 102 and the support 104 may be slightly curved to conform to the shape of the outer leg or anterior lateral aspect of a foot. In one embodiment, linings 108, 110 may be made of a slip resistant material which helps the brace stay in place during movement.

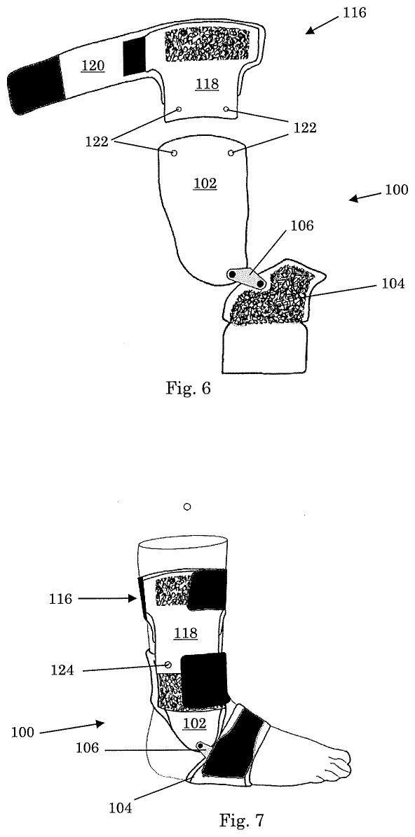

Referring to FIGS. 6 and 7, the subtalar brace 100 may be used with a calf cuff 116. The calf cuff 116 provides support to the ankle and leg and may be removed depending on the injury. The calf cuff 16 is comprised of an outer shell 118 and a removable liner 120. The calf cuff 116 may be attached to the upper end of the lateral piece 102 at attachment points 122 by removable fasteners 124. The removable lining 120 adheres to the inside of the outer shell 118. When used with the subtalar brace 100, the calf cuff 116 wraps around the mid-calf for additional ankle support.

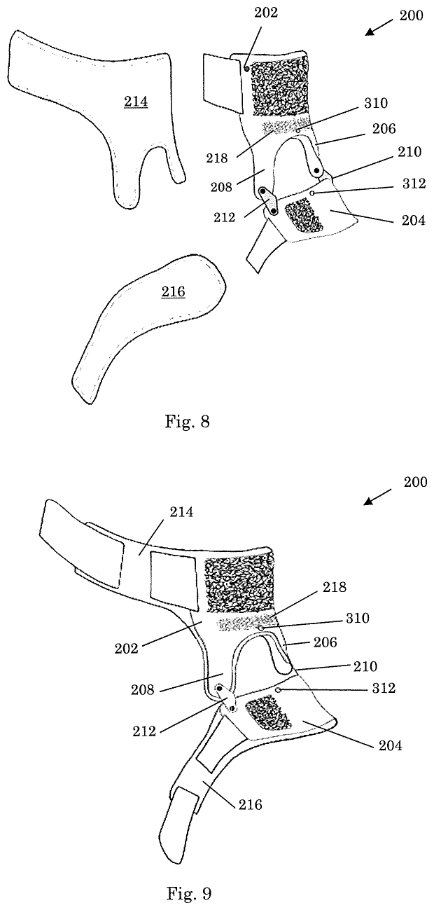

Now, turning to FIGS. 8 and 9, a right-footed dorsum ankle brace 200 according to the Modular Ankle Brace System is shown. The dorsum brace 200 is affixed to the anterior portion of the lower leg and dorsum of the foot to stabilize the ankle while allowing for plantarflexion and dorsiflexion of the ankle and foot. The dorsum brace 200 includes a tibial cuff 202 and a dorsum cuff 204. The tibial cuff 202 and the dorsum cuff 204 may be curved or semi-flexible to conform to the shape of the anterior lower leg and the dorsum of the foot.

In one embodiment, the tibial cuff 202 has a medial stirrup 206 and a lateral stirrup 208 extending downward from the lower end of the tibial cuff 202. The tibial cuff 202 is connected to the dorsum cuff 204 by two dual-hinged brackets 210, 212. The medial bracket 210 connects a medial side of the dorsum cuff 204 to the medial stirrup 206 of the tibial cuff 202. The lateral bracket 212 connects a lateral side of the dorsum cuff 204 to the lateral stirrup 208 of the tibial cuff 202. The position of the medial bracket 210 and the lateral bracket 212 in relation to the tibial cuff 202 and the dorsum cuff 204 gives the dorsum brace 200 the ability to mimic the ankle's natural movements while still providing support to protect against awkward or excessive twisting of the foot during.

The dorsum brace 200 also includes a tibial liner 214 and a dorsum liner 216. The tibial liner 214 attaches to the tibial cuff 202 and wraps around the leg to fasten the tibial cuff 202 to the lower anterior portion of the leg. The dorsum liner 216 attaches to the dorsum cuff 204 and wraps around the mid-foot to secure the dorsum cuff 204 to the dorsum of the foot.

In some embodiments of the Modular Ankle Brace System, the dorsum brace 200 may be used with an immobilizer 300 (not shown) and the tibial cuff 202 and the dorsum cuff 204 may include immobilizer receiver holes 310, 312.

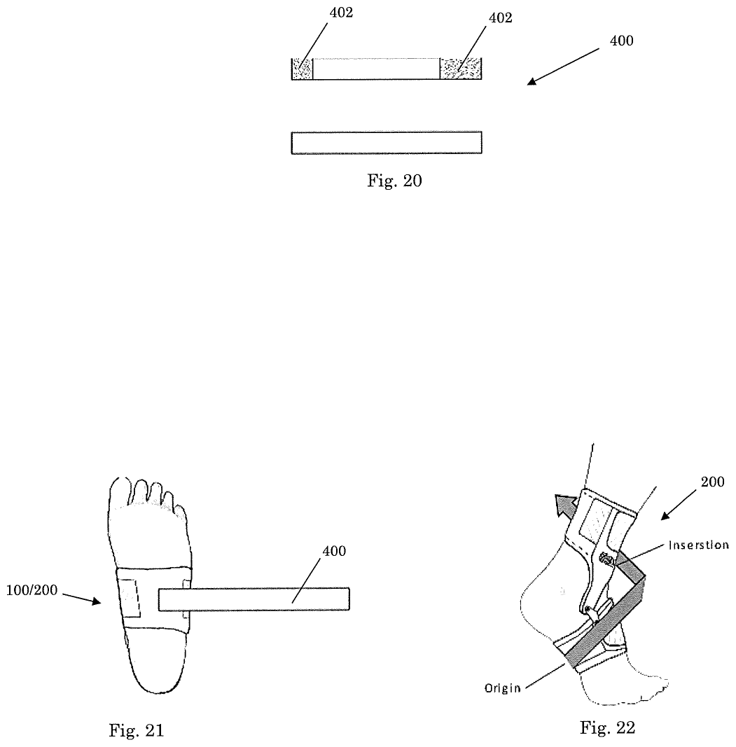

In other embodiments of the Modular Ankle Brace System, the brace 100/200 may be used with a tension strap 400 (shown in FIGS. 20 and 21) and the lateral piece 102 or the tibial cuff 202 may include a tension strap fastening area 218.

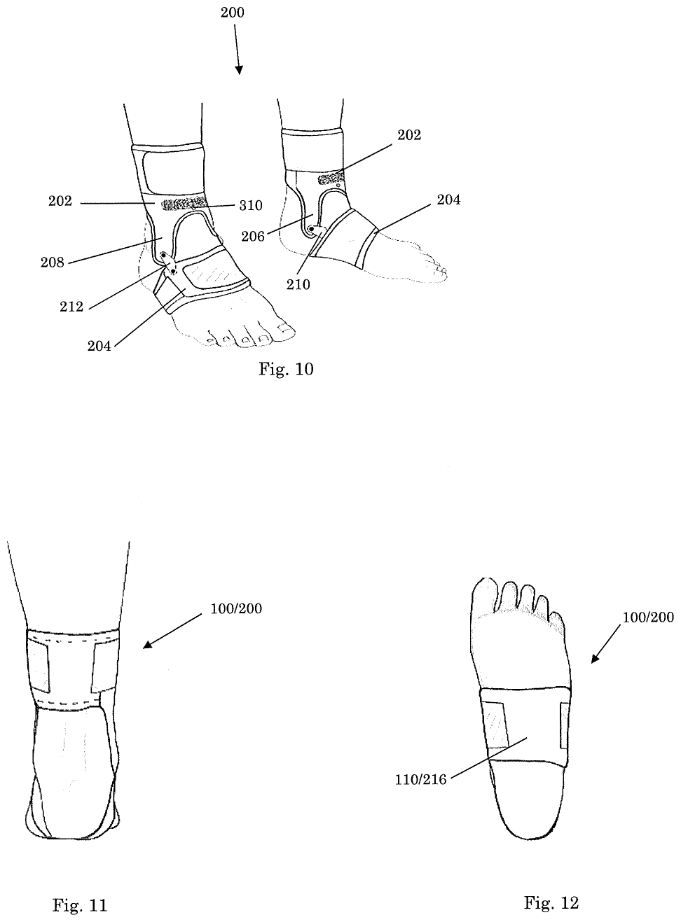

FIG. 10 illustrates the dorsum brace 200 in use on both a left and a right foot showing a lateral and a medial view of the brace 200. The location of the brackets 210, 212 on the stirrups 206, 208 on either side of the dorsum of the foot allows for plantarflexion and dorsiflexion of the ankle by leaving the front aspect of the ankle unimpeded.

Now referring to FIG. 11, a rear view of a foot wearing a brace 100/200 according to the Modular Ankle Brace System is shown. In addition to the exposed and unimpeded anterior aspect of the ankle, the exposed and unimpeded heel and Achilles tendon allow for easier plantarflexion and dorsiflexion.

In FIG. 12, the bottom of a left foot wearing a brace 100/200 according to the Modular Ankle Brace System is shown. This view illustrates another feature of the Modular Ankle Brace System that enhances natural movement of the foot while still protecting the ankle from injury, which is the placement of the support liner 110 or the dorsum liner 216, wrapped around the mid-foot. By wrapping around the mid-foot and limiting the amount of material around the ankle bones, the Modular Ankle Brace System allows for normal movement of the foot and ankle.

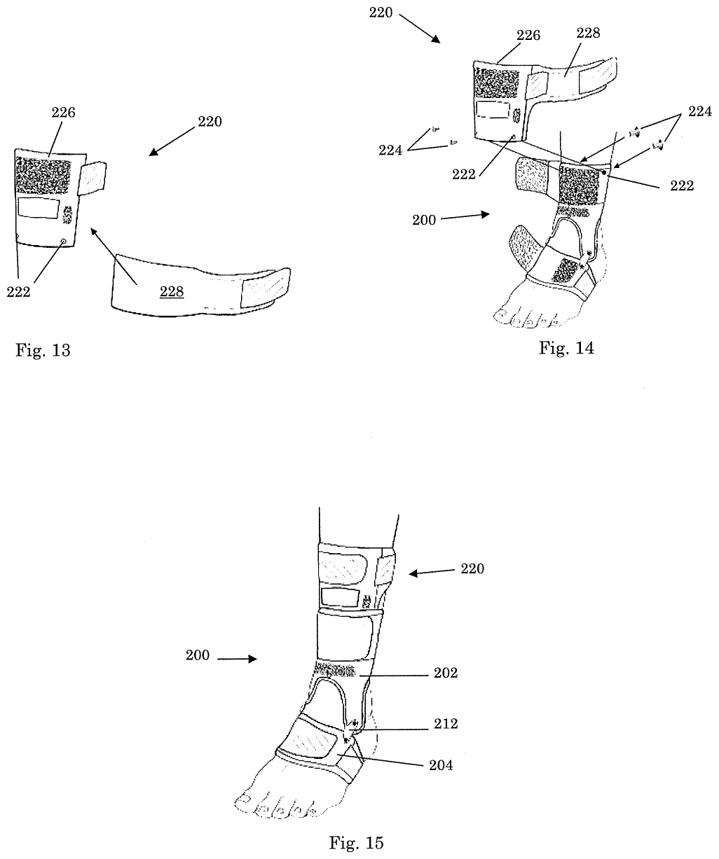

Referring now to FIGS. 13 and 14, in some embodiments, the dorsum brace 200 may be used with an upper tibial cuff 220. The upper tibial cuff 220 may include fastening holes 222 for affixing the upper tibial cuff 220 to the tibial cuff 202 by removable fasteners 224. The upper tibial cuff 220 provides additional support during the early stages of recovery and for permanent injuries. The upper tibial cuff 220 includes an outer shell 226 and an upper tibial liner 228 that attaches to the outer shell 226. The strap of the upper tibial liner 228 wraps around the mid-calf to secure the upper tibial cuff 220 to the shin.

In FIG. 15, a dorsum brace 200 being worn with the upper calf cuff 220 is shown on a left foot.

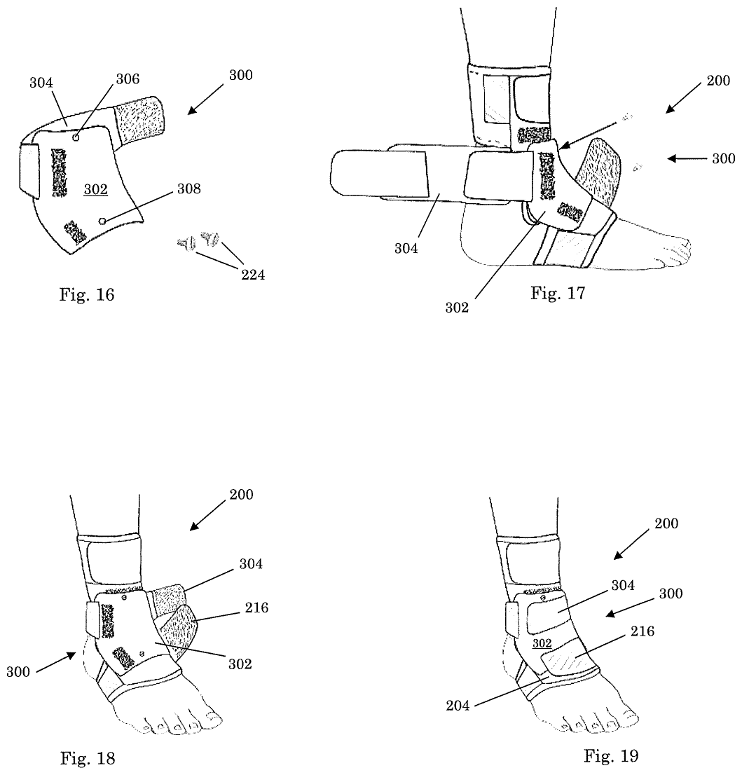

Referring now to FIG. 16, the dorsum brace 200 may be used with an immobilizer 300. The immobilizer 300 includes a front piece 302 and a strap 304. In one exemplary embodiment, the immobilizer 300 is made of a substantially rigid material. Then, the immobilizer 300 may be placed in front of the exposed front of the ankle and the strap 304 wrapped around the ankle to prevent plantarflexion and dorsiflexion. The immobilizer 300 may be used on a new ankle injury until diagnosed by a physician and may be removed based on the severity or progress of the ankle injury.

In one exemplary embodiment, the front piece 302 has a tibial attachment hole 306 and a dorsum attachment hole 308 for fastening the immobilizer 300 to the dorsum brace 200 via removable fasteners 224 to the tibial receiver hole 310 and the dorsum receiver hole 312 (shown in FIGS. 8 and 9).

Referring now to FIGS. 20 to 25, the braces 100/200 may be used with a tension strap 400. The tension strap 400 has fasteners 402 on its ends as shown in FIG. 20. In use, one fastener 402 of the tension strap 400 is attached to the support liner 110 or the dorsum liner 216 on the bottom of the foot as shown in FIG. 21. Then various wrapping techniques may be used depending on if a particular injury requires more support in a specific area. Several wrapping techniques are shown in FIGS. 22 to 25 with the origin, or attachment point, shown and the arrow illustrating the direction of wrapping. Then, the other fastener 402 may be affixed to the tension strap landing 218. The tension strap 400 uses the rigid tibial cuff 202 or lateral piece 102 as leverage to support injured ligaments. The tension strap 400 can also be used to correct over-pronation.

Many different embodiments have been disclosed herein, in connection with the above description and the drawings. It will be understood that it would be unduly repetitious and obfuscating to literally describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

It will be appreciated by persons skilled in the art that the embodiments described herein are not limited to what has been particularly shown and described herein above. In addition, unless mention was made above to the contrary, it should be noted that all of the accompanying drawings are not to scale. A variety of modifications and variations are possible in light of the above teachings.

What is claimed is:

1. A modular ankle brace, the brace comprising:
a lateral piece positioned over a lateral aspect of an ankle and secured to the ankle;
a support positioned over an anterior lateral aspect of a foot and secured to the foot; and
a bracket having a first and second end, the first end of the bracket attached to the lateral piece and the second end attached to the support.
